(19) United States
(12) Patent Application Publication
(10) Pub. No.: US 2011/0305671 A1
(43) Pub. Date: Dec. 15, 2011

(54) Cell Compositions and Methods for Hair Follicle Generation

(75) Inventors: Antonio Armani, Richmond Hill (CA); Sara Armani, Richmond Hill (CA); Charitha Seneviratne, Mississauga (CA); Reza Nazari, Richmond Hill (CA)

(73) Assignee: ALVI ARMANI GENOMICS INC., Richmond Hill (CA)

(21) Appl. No.: 13/083,182

(22) Filed: Apr. 8, 2011

Related U.S. Application Data
(60) Provisional application No. 61/353,272, filed on Jun. 10, 2010.

(51) Int. Cl. A61K 35/36; A61P 17/14

(52) U.S. Cl. 424/93.3

(57) ABSTRACT

The application describes a method of generating a hair follicle, comprising administering a composition comprising epidermal matrix cells, dermal papilla cells, dermal sheath cells and outer root sheath cells to the scalp of a mammal. The composition may optionally be administered with a hair growth agent and/or a carrier. The application also describes a composition comprising epidermal matrix cells, dermal papilla cells, dermal sheath cells and outer root sheath cells and the use of the composition to generate hair follicles.

### CROSS REFERENCE TO RELATED APPLICATIONS

This non-provisional application claims priority from U.S. provisional application US 61/353,272 filed on Jun. 10, 2010, which is incorporated herein by reference in its entirety.

### FIELD

The application relates to compositions and methods for generating hair follicles. In particular, the application relates to implanting in vitro-cultivated hair follicle cells into a scalp to generate and rejuvenate hair follicles in a subject suffering from hair loss.

### BACKGROUND

Hair loss affects millions of people, including over 40% of men over the age of 30. Numerous factors can cause hair loss, including genetic predisposition, autoimmune reactions, scarring, diseases and infection. Hair loss can ultimately lead to complete baldness.

Alopecia is a medical condition in which hair is lost from an area of the body. One symptom of alopecia is hair follicle miniaturization (described below). Alopecia includes both androgenetic alopecia, also known as male pattern baldness, and alopecia areata, which is thought to be an autoimmune disorder.

Normally, a hair follicle cycles through phases including the anagen (growth) phase, the catagen (transition) phase and the telogen (resting or quiescent) phase. In the miniaturization process, the hair follicle enters a prolonged lag phase following the telogen stage. With successive anagen cycles, the follicles become smaller, leading to shorter, finer hair. The miniaturized follicle eventually produces a tiny hair shaft that is cosmetically insignificant. Ultimately, the follicle can stop producing a hair shaft altogether and the area of hair loss can become completely devoid of hair.

Several methods for treating hair loss are available, including drugs such as topical minoxidil and orally-delivered propecia. However, these treatments have achieved limited success in restoring natural hair growth and are only effective while the drugs are being taken.

One surgical treatment for hair loss is hair follicle transplantation, a procedure in which hair follicles are transplanted from a non-balding region of the scalp to a region of hair loss.

Alternatives to hair follicle transplantation are cell-based therapies whereby cells are implanted with the goal of developing new hair follicles. For example, U.S. Pat. No. 4,919,664 to Oliver et al. relates to a method of stimulating hair growth in the skin of a mammal by culturing at least one lower follicular dermal cell, and implanting the cultured cells in the epidermis. U.S. Pat. No. 6,399,057 to Gho describes a method of regenerating hair by: (1) removing hair in the anagen phase, (2) culturing hair follicle cells, and (3) implanting the cultured cells into bald regions. In addition, U.S. Patent Application No. 2007/0128172 to Yoshizato et al. describes the transplantation of cultured dermal papilla cells, dermal sheath cells and epidermal cells into the skin to regenerate hair. In Wu et al. (2006), cultured dermal papilla cells were mixed with outer root sheath cells and transplanted on the dorsal skin of nude mice to induce hair follicle and hair fiber formation.

There remains a need for efficient, cell-based therapies for rejuvenating miniaturized hair follicles and generating new hair follicles.

### SUMMARY OF THE DISCLOSURE

The invention relates to a method of generating a hair follicle in or on the scalp of a mammal, comprising administering a composition comprising epidermal matrix cells, dermal papilla cells, dermal sheath cells and outer root sheath cells to the scalp. The present invention describes a straightforward, efficient and high output method for the isolation of the hair follicle cells, optionally from a single hair follicle. The cell types are readily expanded in vitro, then administered to the scalp of a patient.

In one embodiment of the method, the ratio of dermal papilla cells to dermal sheath cells is 15:1 to 1:1, the ratio of outer root sheath cells to epidermal matrix cells is 15:1 to 1:1, and the ratio of dermal papilla cells plus dermal sheath cells to outer root sheath cells plus epidermal matrix cells is 10:1 to 0.5:1. In an optional embodiment, the ratio of dermal papilla cells to dermal sheath cells is 10:1, the ratio of outer root sheath cells to epidermal matrix cells is 10:1 and the ratio of dermal papilla cells plus dermal sheath cells to outer root sheath cells plus epidermal matrix cells is 1:1.

In another embodiment, the composition comprises 1-3×10⁴ dermal papilla cells, 1-3×10⁴ outer root sheath cells, 1-3×10³ dermal sheath cells and 1-3×10³ epidermal matrix cells. In an optional embodiment, the composition comprises 2×10⁴ dermal papilla cells, 2×10⁴ outer root sheath cells, 2×10³ dermal sheath cells and 2×10³ epidermal matrix cells.

In another embodiment of the invention, the cells are isolated from a native hair follicle or from a plurality of native hair follicles. The cells may be isolated by microdissection, enzymatic treatment or a combination of microdissection and enzymatic treatment.

In another embodiment of the method, each type of cel of the composition is expanded separately in vitro prior to administration. In a further embodiment, each cell type is expanded in media specific to the cell.

In one embodiment, the epidermal matrix cells are cultured in culture medium comprising antibiotic, antimycotic, human recombinant bFGF, human insulin, hydrocortisone, and bovine pituitary extract. Optionally, the culture medium further comprises phorbol myrsitate acetate.

In another embodiment, the outer root sheath cells are cultured in culture medium comprising antibiotic, antimycotic, human recombinant EGF, human insulin, hydrocortisone, epinephrine, human transferrin and bovine pituitary extract.

In another embodiment, the dermal papilla cells are cultured in medium comprising FBS, L-glutamine, antibiotic, antimycotic, human recombinant bFGF, human recombinant Wnt-3 and human recombinant BMP6.

In another embodiment, the dermal sheath cells are cultured in medium comprising FBS, L-glutamine, antibiotic, antimycotic, human recombinant bFGF and human recombinant PDGF-AA.

In another aspect of the invention, the dermal papilla cells are aggregated into at least one sphere prior to implantation. In a further aspect, the sphere is formed by microencapsulation or centrifugation. In another aspect, there are 100 to 100,000 dermal papilla cells in one sphere. In another aspect, there are 10,000 to 20,000 dermal papilla cells in one sphere. In yet another aspect, the spheres further comprise dermal sheath, epidermal matrix and/or outer root sheath cells.

In one embodiment of the invention, the composition is administered to a native hair follicle such that the cells contact the native hair follicle. Optionally, the native hair follicle is a miniaturized hair follicle. In another embodiment, the composition is administered to an incision in the scalp. In a further embodiment, the composition is administered directly to the scalp, optionally between the dermis and the epidermis.

In another embodiment of the invention, the composition further comprises at least one hair growth agent. In another embodiment of the composition, the composition further comprises at least one hair growth agent. In a further embodiment, the at least one hair growth agent is selected from the group consisting of: IGF-1, FGF-2, FGF-10, PDGF-AA, Wnt-3a, noggin, ephrin-A3, SHH, BMP-6 and hypoxanthine.

In one embodiment of the method, the composition comprises IGF-1, FGF-2, PDGF-AA, Wnt-3a, noggin, BMP-6 and hypoxanthine. In another embodiment, the composition comprises IGF-1, FGF-2, FGF-10, PDGF-AA, Wnt-3a, noggin, ephrin-A3, SHH, BMP-6 and hypoxanthine. In yet another embodiment, the composition comprises FGF-2, Wnt-3a, SHH, and hypoxanthine.

In one embodiment of the invention, the composition is administered by injection into the scalp. In another embodiment, the composition is administered by 10 to 50 injections per square centimeter of the scalp. In another aspect of the invention, each injection contains 1 microliter to 100 microliters of the composition. In a further aspect, each injection contains 10 to 20 microliters of the composition.

In one embodiment of the invention, the composition is incorporated in a carrier prior to administration. In another embodiment, the carrier is a biomatrix. In a further embodiment, the biomatrix comprises hyaluronic acid.

The invention also relates to a composition comprising epidermal matrix cells, dermal papilla cells, dermal sheath cells and outer root sheath cells.

In one embodiment of the composition, the dermal papilla cells are formed into at least one sphere. In another embodiment of the method, the ratio of dermal papilla cells to dermal sheath cells is 15:1 to 1:1, the ratio of outer root sheath cells to epidermal matrix cells is 15:1 to 1:1, and the ratio of dermal papilla cells plus dermal sheath cells to outer root sheath cells plus epidermal matrix cells is 10:1 to 0.5:1. In an optional embodiment, the ratio of dermal papilla cells to dermal sheath cells is 10:1, the ratio of outer root sheath cells to epidermal matrix cells is 10:1 and the ratio of dermal papilla cells plus dermal sheath cells to outer root sheath cells plus epidermal matrix cells is 1:1.

In another embodiment, the composition comprises 1-3×10⁴ dermal papilla cells, 1-3×10⁴ outer root sheath cells, 1-3×10³ dermal sheath cells and 1-3×10³ epidermal matrix cells. In an optional embodiment, the composition comprises 2×10⁴ dermal papilla cells, 2×10⁴ outer root sheath cells, 2×10³ dermal sheath cells and 2×10³ epidermal matrix cells.

In another embodiment of the composition, the composition further comprises at least one hair growth agent. In a further embodiment, the at least one hair growth agent is selected from the group consisting of: IGF-1, FGF-2, FGF-10, PDGF-AA, Wnt-3a, noggin, ephrin-A3, SHH, BMP-6 and hypoxanthine.

In one embodiment of the composition, the composition comprises IGF-1, FGF-2, PDGF-AA, Wnt-3a, noggin, BMP-6 and hypoxanthine. In another embodiment, the composition comprises IGF-1, FGF-2, FGF-10, PDGF-AA, Wnt-3a, noggin, ephrin-A3, SHH, BMP-6 and hypoxanthine. In yet another embodiment, the composition comprises FGF-2, Wnt-3a, SHH, and hypoxanthine.

In another embodiment of the composition, the composition comprises a carrier. In another embodiment, the carrier is a biomatrix. In a further embodiment, the biomatrix comprises hyaluronic acid.

The invention also relates to the use of the composition for the generation of hair follicles or the rejuvenation of a hair follicle, optionally a native hair follicle.

### BRIEF DESCRIPTION OF THE DRAWINGS

Embodiments of the invention will be shown in relation to the drawings in which the following is shown:

FIG. 1. ORS cells cultured in keratinocyte growth medium one day (A) and seven days (B) after cell isolation.

FIG. 2. EM cells and melanocytes cultured in melanocyte growth medium one day (A and B), five days (C) and eight days (D) after isolation.

FIG. 3. (A) Dermal papilla isolated by the enzymatic method and placed in supplemented DMEM. (B) DP cells growing out of the attached dermal papilla 2 days after isolation and maintained in supplemented DMEM. (C) DP cells after 7 days of culture in the same medium. (D) DP cells growing close to complete confluence after 10 to 14 days cultured in the same medium.

FIG. 4. (A) DS cells 3 days after isolation by enzymatic method and maintained in supplemented DMEM. (B) DS cells after 10 days culture in the same medium. (C) DS cells growing to the complete confluence after 20 days cultured in the same medium.

FIG. 5. DP sphere formed from 10⁴ DP cells in a round-bottom well of a 96-well Low-Cell Binding plate one day after seeding.

FIG. 6. Comparison of hair density per cm² in the left temporal area of the scalp (solid line) following injection of cells and growth composition with hair density in the right temporal area of the scalp (dotted line) following injection of cells and growth composition and hyaluronic acid.

FIG. 7. Depiction of hair growth in the left temporal area (A and B), in which cells and growth factors were injected (without hyaluronic acid gel) compared to that of the right temporal (C and D) areas, in which cells and growth factors were mixed in hyaluronic acid gel before being injected. The pictures were taken before the injections at week 0 (FIGS. 7A and 7C) and after 3 months (FIGS. 7B and 7D). In FIG. 7D, the hairs are stained for better visualizing. The new hairs are indicated by arrows.

### DETAILED DESCRIPTION OF THE INVENTION

The application relates to a method of generating a hair follicle in a mammal, such as a human. The mammal is typically suffering from hair loss, such as chronic hair loss. The method comprises administering a composition comprising epidermal matrix (EM) cells, dermal papilla (DP) cells, dermal sheath (DS) cells and outer root sheath (ORS) cells to the scalp of a mammal. In one embodiment of the invention, each of the cell types are isolated from a single hair follicle or a plurality of hair follicles, and expanded separately in cell culture. The four cell types are combined and optionally mixed with a growth composition and/or a carrier. The four cell types are also readily administered separately or in other combinations. The final composition is typically injected into the scalp, for example, into miniaturized hair follicles or into incisions in the scalp. The final composition may also be injected directly into the scalp, typically between the dermis and the epidermis.

In the present application, the term “hair follicle” refers to a tube-like opening in the epidermis where the hair shaft develops. The present invention relates to native hair follicles and formed hair follicles. As used herein, a native hair follicle is a pre-existing, naturally occurring hair follicle in a scalp. A native hair follicle may be a miniaturized hair follicle, as described below. A native hair follicle typically includes the following structures: papilla, matrix, root sheath, sebaceous gland and hair fiber (also known as a hair shaft). The hair shaft may be of decreased diameter or not present at all, depending on the extent of alopecia. Formed hair follicles are hair follicles formed as new hair follicles by administration of the cell compositions described herein. As used herein, a formed hair follicle optionally includes a plurality of the following structures: papilla, matrix, root sheath, sebaceous gland and hair fiber (also known as a hair shaft). The hair shaft may be not present at all, depending on the extent and stage of the formed follicle development.

A “miniaturized hair follicle” refers to a hair follicle that has undergone miniaturization as a result of progressive hair loss. Miniaturized hair follicles are no longer cycling normally, but rather enter a prolonged lag phase following a telogen stage. With successive anagen cycles, the follicles become smaller, leading to a shorter, finer hair that is cosmetically insignificant (vellus hair). Specifically, vellus hairs produced from a miniaturized hair follicle often have a diameter of less than 0.04 mm. In contrast, terminal hairs (long, darkly pigmented hairs) are generally over 0.06 mm in diameter and intermediate hairs, which share characteristics of both vellus hairs and terminal hairs, are typically between 0.04 and 0.06 mm in diameter.

In the present invention, the term “generating” refers to i) generating a new hair follicle (a formed hair follicle) in the skin where no follicle existed before, or ii) rejuvenating an existing hair follicle (a native hair follicle) such as a miniaturized hair follicle. A hair follicle that has been generated or rejuvenated may or may not include a hair fiber.

In the typical, untreated healthy native hair follicle, epidermal matrix, dermal papilla, dermal sheath and outer root sheath cells each play a role in forming the hair follicle and producing the hair shaft. Without wishing to be bound by theory, the four cell types are described briefly below:

The dermal papilla (DP) is a group of specialized dermal fibroblast cells derived from the embryonic mesoderm. During embryogenesis, the establishment of a DP helps develop hair follicles and associated modified structures like sebaceous glands. The DP cells begin to aggregate in the dermis just below the epidermis. Above the dermal papilla, an epidermal plug, or peg, of cells develops and proliferates, growing into the dermis towards the dermal papilla. The mesoderm-derived dermal papilla and the ectoderm-derived epidermal plug communicate via molecular signals with the result of further proliferation of epidermal matrix cells and differentiation into the various sheaths and hair fibre structures. Thus the development of a hair follicle involves a continuum through induction, initiation, elongation and differentiation stages (Oliver and Jahoda, 1988).

Dermal sheath (DS) cells are considered to be the progenitors of DP cells, which can be transformed into papilla cells to form new papilla for maintaining the size of hair follicle (Oliver, 1991).

Upper (superior) outer root sheath (ORS) cells, located in the bulge of human hair follicles, have high proliferative potential and ability to differentiate (Wu et al., 2006). In the telogen hair follicle, DP cells come into close proximity with bulge ORS cells, resulting in the induction of these cells. Upon induction, the ORS cells migrate to the bulb and establish the infrastructure of a new hair shaft in the anagen phase (Botchkarev and Kishimoto, 2003). There, they differentiate to epidermal matrix (EM) cells. The EM cells are in direct communication with DP cells for proliferation and differentiation and play an important role in hair shaft production. EM cells differentiate into the cells that make all layers of the hair follicle as well as keratinocytes (Botchkarev and Kishimoto, 2003).

### Hair Follicle Collection and Isolation

According to the methods of the invention, native hair follicles are collected from any part of the body of the cell donor. In one embodiment, hair follicles are collected from the back of the donor's head, optionally the occipital area of the scalp. In another embodiment, hair follicles are collected from at least one side of the donor's head.

The follicles are isolated optionally from one single donor or from a plurality of different donors. Optionally, the hair follicles are collected from the donor who will ultimately be implanted with the cell composition (autologous cell donor for autologous cell transplant).

Native hair follicles are collected or removed by any extraction method known in the art. In one aspect of the invention, various plucking or surgical methods are employed including FUS (follicular unit strip) and FUE (follicular unit extraction). The donor skin is disinfected prior to hair follicle extraction by any routine surgical disinfectant, including but not limited to betadine, hydrogen peroxide or alcohol.

The isolated hair follicles are disinfected by highly concentrated antibiotics and antimycotics, including, but not limited to, penicillin G, streptomycin and/or amphotericin B. In one embodiment, the concentration of the antibiotics is between 3- to 10-fold of the basic antibiotic solutions (100 units/ml penicillin G, 100 ug/ml streptomycin and 0.25 ug/ml amphotericin B). The high concentration antibiotic-antimycotic solutions may be prepared in any isotonic solutions, including saline, phosphate buffered solution (PBS), Hanks' balanced salt solution (HBSS), etc., containing or lacking Ca2+ and Mg2+. The exposure time to disinfectant solution can be one 1 minute or up to 30 minutes or longer. The hair follicles are maintained in the same isotonic solution or a nutritive solution, such as a defined cell culture medium, until starting the cell isolation process. The solution or medium is optionally supplemented with various growth factors, growth hormones, and/or growth stimulants.

### Cell Isolation and Expansions

In one embodiment of the invention, the different cell types (for example, DP, DS, ORS, and EM cells) are isolated from a single hair follicle. In another embodiment of the invention, the cell types are isolated from less than 100 hair follicles, optionally less than 30 follicles and optionally less than 10 hair follicles. In a further embodiment, the cell types are isolated from up to 500 hair follicles.

The different cell types are optionally isolated from hair follicles using a variety of methods. In one embodiment of the invention, the cell types are isolated by microdissection, enzymatic treatment or a combination of the two methods.

In microdissection, typically the bulb, epidermal matrix, sheaths and shaft of each hair follicle are separated under a binocular using microdissection fine tools. The hair follicles and separated parts are bathed in isotonic solutions or in specific cell culture media. Optionally, the bulb is maintained in DP-specific cell culture medium, the sheaths in keratinocyte-specific medium, and the epidermal matrix in the melanocyte-specific medium. Each of the said parts is further dissected into pieces and components. In one embodiment, the epidermal matrix is teased off and collected from around the DP, the DP is cut off the bulb and the three parts are maintained in separate vessels. Collected similar parts from several hair follicles may be cultivated.

In enzymatic treatment method, the hair follicles are exposed to tissue-digesting enzymes including, but not limited to, Trypsin-EDTA, Dispase, Collagenase (any type), TrypLE Express, etc. The enzymatic method is optionally followed with a method whereby the tissue is passed through meshes to further isolate the cells.

In combination methods, hair follicles are partially microdissected prior to, during, and/or after exposure to enzymes. The combination methods increase isolation efficacy by minimizing time and labour.

Once isolated, the different cell types are cultivated and expanded in general or specific cell-culture media. The media is optionally commercially prepared or prepared by the user. Optionally, the media is specifically designed and produced for a specific type of cell through the addition of supplements, such as growth factors, vitamins, etc. to any cell-specific or basic medium. The supplements may be of animal or human sources or produced via recombinant technology. The supplements optionally include serum, gland extracts (such as pituitary extracts), growth hormone(s), growth factor(s), signalling molecule(s), any type of stimulatory or inhibitory small or large molecule(s), ligand(s), nucleic acid(s), chemical compounds(s), antibodies, antibiotics, drug(s), etc. In another aspect of the invention, the cells are cultured in conditioned media.

In one embodiment of the invention, the EM cells are cultured in culture medium containing human recombinant bFGF, human insulin, hydrocortisone, bovine pituitary extract with or without phorbol myrsitate acetate.

In another embodiment, the ORS cells are cultured in culture medium containing human recombinant EGF, human insulin, hydrocortisone, epinephrine, human transferrin and bovine pituitary extract.

In another embodiment, the DP cells are cultured in medium containing FBS, L-glutamine, antibiotics, human recombinant bFGF, human recombinant Wnt-3 and human recombinant BMP6.

In another embodiment, the DS cells are cultured in medium containing FBS, L-glutamine, antibiotics, human recombinant bFGF and human recombinant PDGF-AA.

Optionally, the cells are sub-cultured for several passages. Optionally, the cells are passaged for at least: 2 passages, 3 passages, 5 passages or 10 passages before being combined into the final composition for injection. The cells at different passages may be frozen for future use and any commercially available or lab-prepared cell-freezing solutions may be used for this procedure. The frozen cells are optionally revived and re-cultured before being injected into the scalp, according to any reviving method known in the art.

The initial cultures and/or sub-cultures of cells are optionally performed in un-treated, tissue culture-treated, collagen type I-coated, collagen type IV-coated, or any coated tissue culture flask or Petri dish or multi-well plate, or any other tissue culture vessel.

### Dermal Papillae Cell Sphere Formation

In one aspect of the invention, the DP cells are aggregated into spheres prior to mixing with the other cell types. In one embodiment, the DP spheres are formed by aggregating DP cells by microencapsulation (Li et al., 2005), by centrifugation in round-bottom multi-well plates, or by culturing in low cell binding multi-well plates (Osada et al., 2007), in culture media mixed with methylcellulose in multi-well plates, or in droplets hanging from the lid of a Petri dish or microscopic glass slide or cover slips.

The number of DP cells required to form spheres optionally varies from at least 100 cells, optionally at least 300 cells, up to, for example, 10,000 or 25,000 cells, or 10,000 to 20,000 cells. In one embodiment of the invention, the spheres are composed of only DP cells. In another embodiment, the spheres contain a mixture of DP and DS cells. While various ratios of DP:DS cells are contemplated, in one aspect of the invention, the ratio is within the range of 100:1 to 1:1, optionally 20:1 to 5:1, optionally 10:1. In a further embodiment, the spheres also include ORS cells and/or EM cells. The ORS and EM cells are mixed with the DP and DS cells before forming the spheres or added when the spheres are formed. The ratio of ORS and/or EM cells in the spheres may vary, with an optimum ratio of ORS and/or EM cells:DP cells in the range of 1:100 to 1:1, optionally 1:10 for EM:DP cells and optionally 1:1 for ORS:DP cells. The spheres may be used immediately after being formed or may be cultured for several days before being used.

### Cell Composition

In one embodiment, the cell composition comprises dermal papilla cells, outer root sheath cells, dermal sheath cells and epidermal matrix cells. Optionally, the composition comprises any combination of the following: dermal papilla cells, outer root sheath cells, dermal sheath cells and epidermal matrix cells. In another embodiment, the cell composition comprises additional cell types, optionally additional hair follicle cell types.

In one embodiment of the method, the ratio of dermal papilla cells to dermal sheath cells is 50:1 to 0.5:1, optionally 15:1 to 1:1, optionally 10:1 to 1:1, the ratio of outer root sheath cells to epidermal matrix cells is 50:1 to 0.5:1, optionally 15:1 to 1:1, optionally 10:1 to 1:1, and the ratio of dermal papilla cells plus dermal sheath cells to outer root sheath cells plus epidermal matrix cells is 50:1 to 0.5:1, optionally 10:1 to 0.5:1. In an optional embodiment, the ratio of dermal papilla cells to dermal sheath cells is 20:1 to 5:1, optionally 10:1, the ratio of outer root sheath cells to epidermal matrix cells is 20:1 to 5:1, optionally 10:1 and the ratio of dermal papilla cells plus dermal sheath cells to outer root sheath cells plus epidermal matrix cells is 2:1 to 0.5:1, optionally 1:1.

In another embodiment, the composition comprises 0.5-5×10⁴ dermal papilla cells, 0.5-5×10⁴ outer root sheath cells, 0.5-5×10⁴ dermal sheath cells and 0.5-5×10⁴ epidermal matrix cells. In another embodiment, the composition comprises 1-3×10⁴ dermal papilla cells, 1-3×10⁴ outer root sheath cells, 1-3×10³ dermal sheath cells and 1-3×10³ epidermal matrix cells. In yet another embodiment, the composition comprises 1.5-2.5×10⁴ dermal papilla cells, 1.5-2.5×10⁴ outer root sheath cells, 1.5-2.5×10³ dermal sheath cells and 1.5-2.5×10³ epidermal matrix cells. In an optional embodiment, the composition comprises 2×10⁴ dermal papilla cells, 2×10⁴ outer root sheath cells, 2×10³ dermal sheath cells and 2×10³ epidermal matrix cells.

### Growth Promoting Composition

According to one embodiment of the invention, the cells and/or DP spheres are mixed with a growth promoting composition prior to administration. The term “growth promoting composition” refers to any composition that increases or promotes the growth of hair, hair follicle cells (for example, EM, DP, DS or ORS cells) and/or explant hair follicles. The growth promoting composition may contain one or more hair growth agents as defined below.

The term “hair growth agent” refers to any protein, nucleic acid, polysaccharide or lipid that is associated with increasing, promoting or maintaining the growth of hair, hair follicles or hair follicle cells. For example, hair growth agents can include growth stimulants, such as growth hormones, signalling molecules, chemokines/cytokines involved in wound healing, stimulatory or inhibitory small or large molecules, ligands, nucleic acids, chemical compounds, antibodies, drugs, plant extracts or their fractions, stem cell mobilizing factors from plant extracts or fractions, etc.

In one embodiment of the invention, a hair growth agent is a protein, optionally a cellular growth factor. In another embodiment of the invention, a hair growth agent is hypoxanthine, a naturally occurring purine derivative.

The term “cellular growth factor” refers to a naturally occurring substance capable of stimulating cellular growth, proliferation and differentiation. Examples of growth factors that play a role in hair follicle development include, but are not limited to: IGF-1 (insulin-like growth factor-1), FGF-2 (fibroblast growth factor-2 (basic)), FGF-10, PDGF-AA (platelet-derived growth factor-AA), Wnt-3a, Noggin, Ephrin-A3, SHH (sonic hedgehog) and BMP-6 (bone morphogenesis protein-6).

### Carrier for Injection

In a further embodiment of the invention, the cells and/or spheres and/or growth promoting composition are injected in conjunction with a vehicle. In this embodiment, an effective quantity of the active substance(s) is combined in a mixture with a pharmaceutically acceptable vehicle. Suitable vehicles are described, for example, in Remington's Pharmaceutical Sciences (2003-20^{th} Edition). On this basis, the compositions include, albeit not exclusively, solutions of the substances in association with one or more pharmaceutically acceptable vehicles or diluents, and contained in buffered solutions with a suitable pH and iso-osmotic with the physiological fluids.

Pharmaceutical compositions include, without limitation, lyophilized powders or aqueous or non-aqueous sterile injectable solutions or suspensions, which optionally further contain antioxidants, buffers, bacteriostats and solutes that render the compositions substantially compatible with the tissues or the blood of an intended recipient. Other components that are optionally present in such compositions include, for example, water, surfactants (such as Tween™), alcohols, polyols, glycerin and vegetable oils. Extemporaneous injection solutions and suspensions may be prepared from sterile powders, granules, tablets, or concentrated solutions or suspensions. The composition can be supplied, for example but not by way of limitation, as a lyophilized powder which is reconstituted with sterile water or saline prior to administration to the subject.

Suitable pharmaceutically acceptable carriers include essentially chemically inert and nontoxic compositions that do not interfere with the effectiveness of the biological activity of the pharmaceutical composition. Examples of suitable pharmaceutical carriers include, but are not limited to, water, saline solutions, glycerol solutions, ethanol, N-(1(2,3-dioleyloxy)propyl)N,N,N-trimethylammonium chloride (DOTMA), diolesylphosphotidyl-ethanolamine (DOPE), and liposomes. Such compositions should contain a therapeutically effective amount of the compound(s), together with a suitable amount of carrier so as to provide the form for direct administration to the subject.

In a further embodiment, the carrier is in the form of a gel, bead, foam or sponge or any other semi-solid form known in the art.

In one embodiment of the invention, the carrier solidifies before injection (i.e. outside the body), in an alternate embodiment, the carrier solidifies after injection (i.e. inside the body). In a further embodiment, the carrier is made from various bio-tolerated, bio-compatible, and/or bio-degradable materials, such as any type of peptide hydrogel, matrix gel, agarose, cellulose, methylcellulose, gelatine, collagen, chondroitin-sulphate, chitosan, heparin, peptidoglycan, glycosaminoglycan, hyaluronic acid/hyaluronan, polymers, silica, etc., from any biological and/or synthetic source.

In an optional embodiment of the invention, the carrier is a biomatrix. By the present application, the term “biomatrix” refers to a natural matrix or scaffold, usually made from fibrous macromolecules, on which cells grow to form certain types of tissues. Optionally, the biomatrix is biodegradable and biocompatible. Hyaluronic acid is one example of a biomatrix.

### Injection to Scalp

In one embodiment of the invention, the final composition (four cell types, optionally with a growth composition and optionally with a carrier) is administered to the scalp by injection. The term “administration” describes any suitable means of delivering the cell composition to the subject. In one embodiment, the cell composition is administered by injection. In another embodiment, the composition is administered by surgical implantation. In the process of surgical implantation, the cell composition is implanted using surgical techniques, between the epidermis and the dermis of a subject.

The administration of the final composition into the scalp may be intrafollicular (in the hair follicle) or interfollicular (between hair follicles). In one embodiment of the invention, the composition is administered to a native hair follicle such that the cells contact the native hair follicle. Optionally, the native hair follicle is a miniaturized hair follicle. In another embodiment of the invention, the composition is administered into incisions in the scalp. In a further embodiment, the composition is administered directly into the scalp, optionally between the epidermis and the dermis.

Injection is performed with any type of syringe, such as an insulin syringe, a Hamilton syringe, etc., or a micropipette.

In one aspect of the invention, the number of injections is 5 to 100 injections per square centimeter of the scalp, optionally 10 to 50 injections per square centimeter. In another aspect, 10 or 15 injections per square centimeter are performed. In another aspect of the invention, up to 50 or 60 injections per square centimeter are performed.

According to one embodiment of the invention, 1 microliter to 100 microliters, optionally 5 to 50 microliters, optionally 10 to 20 microliters of composition is injected per injection. In another embodiment of the invention, 1 to 10 DP spheres, optionally 2 to 3 DP spheres are injected.

The present invention allows hair restoration in people with extensive hair loss due to androgenetic alopecia, burns, trauma, chemotherapy, radiation therapy, or scarring alopecia or who otherwise have too little donor hair for traditional transplants. The technology also provides opportunities for high risk people (such as fire fighters, radioactive field workers, high-risk people for cancer, etc.) or patients subjected to chemotherapy or radiation therapy to freeze and save some of their healthy hair follicle cells before hair loss.

### EXAMPLES

Embodiments of the present invention will be illustrated in a non-limiting way by reference to the examples below.

### Example 1: Hair Follicle Collection and Preparation

A male with partial hair loss was selected as a follicle provider after being examined for health. Approximately 100 hair follicles at anagen phase were obtained from the back of the scalp through the follicular unit extraction (FUE) standard follicle extraction procedure. The follicles were soaked in Ca2+- and Mg2+-free PBS (Yoo et al., 2007), containing 5x antibiotics (500 units/ml penicillin G, 500 μg/ml streptomycin and 1.25 μg/ml amphotericin B) for 20 min. The hair follicles were washed once with antibiotic-free PBS.

### Example 2: Hair Follicle Maintenance

Hair follicles were transferred to Petri dishes containing complete Williams' E medium, supplemented with L-glutamine (2 mM), antibiotics/antimycotics (100 units/ml penicillin G, 100 ug/ml streptomycin and 0.25 ug/ml amphotericin B), hydrocortisone (10 ng/ml) (Philpott, Green and Kealey, 1989), and human recombinant IGF-1 (10 ng/ml). The follicles were incubated at 37° C. and 5% CO2/95% air until cell isolation.

### Example 3: Hair Follicle Preparation for Cell Isolation

Hair follicles were transferred to PBS containing dispase and incubated overnight at 4° C. The next morning, the hair follicles were washed with PBS. Each follicle was transected into the bulb, the follicle stem, and the epidermal matrix in a Petri dish containing Hank's Buffered Salt Solution (HBSS).

### Example 4: Isolation and Culture of the ORS Cells

The hair shafts were removed from the epithelial sheath and all attached dermal sheaths were teased off the shaft in the medium. The dermal sheaths were collected by centrifugation and the pellet was resuspended in HBSS containing collagenase type IV (100 U/ml) and incubated at 37° C. until the tissue was partially degraded. The enzymatic reaction was stopped with EDTA and dilution with PBS followed by a high-speed centrifugation for 5 min. The pellet was resuspended in TrypLE Express (Invitrogen) and incubated at 37° C. until the cells were dispersed. The cells were washed with PBS followed by a low-speed centrifugation for 10 min. The pellet was resuspended in culture medium containing human recombinant EGF, human insulin, hydrocortisone, epinephrine, human transferrin and Bovine Pituitary Extract (BPE). To keep the ORS cells in their optimal primary cell state, the medium contained low concentration of CaCl₂, optimally 0.06 to 0.1 mM. The isolated ORS cells were cultured with the medium in a type I collagen-coated multi-well plate at 37° C. and 5% CO₂/95% air. The medium was changed twice a week. When the culture was confluent, the cells were detached with TrypLE Express or Trypsin-EDTA and recultured. The cells were frozen and revived in the Recovery™ Cell Culture Freezing Medium (Invitrogen) according to the manufacturer's protocol.

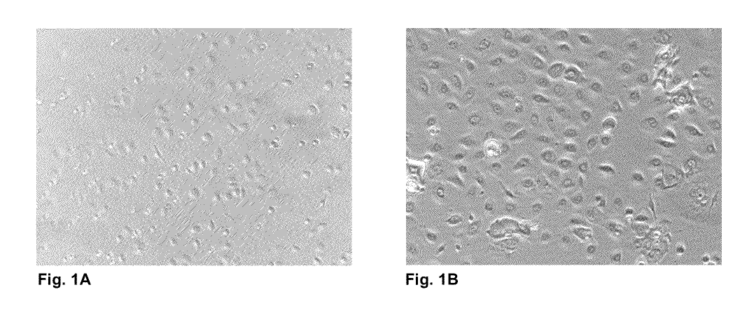

FIG. 1A shows cultured ORS cells one day after isolation (100X magnification) and FIG. 1B shows cultured ORS cells 7 days after isolation (250X magnification). Images were taken with a phase contrast microscope.

### Example 5: Isolation and Culture of the EM Cells

The microdissected epidermal matrix of hair follicles were exposed to Collagenase IV (100 U/ml) in HBSS at 37° C. until the tissue was partially degraded. The enzymatic reaction was stopped with EDTA and dilution with PBS followed by a high-speed centrifugation for 5 min. The pellet was resuspended in TrypLE Express and incubated at 37° C. until the cells were dispersed. The cells were washed with PBS followed by a low-speed centrifugation for 10 min. The pellet was resuspended in a culture medium containing human recombinant bFGF, human insulin, hydrocortisone, Bovine Pituitary Extract (BPE), and phorbol myristate acetate. To keep the EM cells in their primary cell state, culture conditions were optimized for human recombinant SCF to be 5 ng/ml. The isolated EM cells were cultured with such medium in a type I collagen-coated multi-well plate at 37° C. and 5% CO2/95% air. The medium was changed twice a week. When the culture was confluent, the cells were detached with TrypLE Express or Trypsin-EDTA and recultured. The cells were frozen and revived in the Recovery™ Cell Culture Freezing Medium according to the manufacturer's protocol.

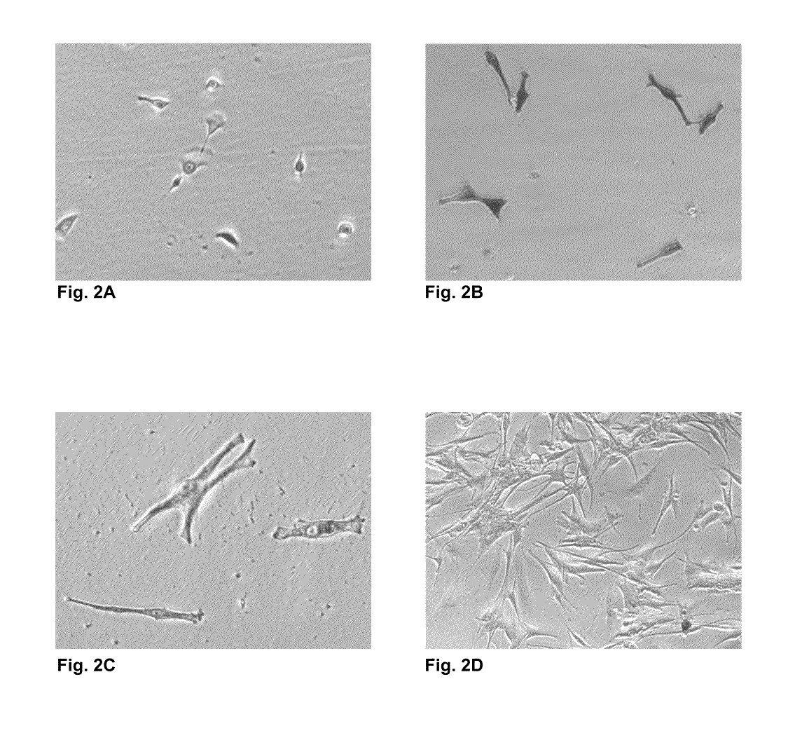

FIG. 2 shows cultured EM cells and melanocytes at day 1 (FIG. 2A and 2B), day 5 (FIG. 2C) and day 8 (FIG. 2D) after isolation. Images were taken at 100X magnification with a phase contrast microscope.

### Example 6: Isolation and Culture of DP and DS Cells

The microdissected bulbs were suspended in HBSS containing collagenase I (200 U/ml) and incubated for 30 min until the DP stalks were digested. The enzymatic reaction was stopped with EDTA and the DPs were released by agitation. The DPs and empty bulbs were separated by differential centrifugation and cultured in especially supplemented DMEM media designed to maintain the in-vivo HF induction capacity of both DP and DS cells. The medium for DP cells contains 20% FBS, 2 mM L-glutamine, antibiotics, human recombinant bFGF (20 ng/ml), human recombinant Wnt-3a (20 ng/ml), and human recombinant BMP6 (400 ng/ml). The medium for DS cells contains 20% FBS, 2 mM L-glutamine, antibiotics, human recombinant bFGF (20 ng/ml), and human recombinant PDGF-AA (10 ng/ml).

The initial cultures of DP and DS cells were carried out in 24-well plates coated with type I collagen. In DP cultures, an average of 5 DPs are seeded per well. The medium was changed once a week when cells were observed growing out of the DP. When the culture was confluent, the cells were passaged with TrypLE Express or Trypsin-EDTA. The serum content of DMEM was reduced to 10% after the first passage. The cells were frozen and revived in the Recovery™ Cell Culture Freezing Medium according to the manufacturer's protocol (Invitrogen).

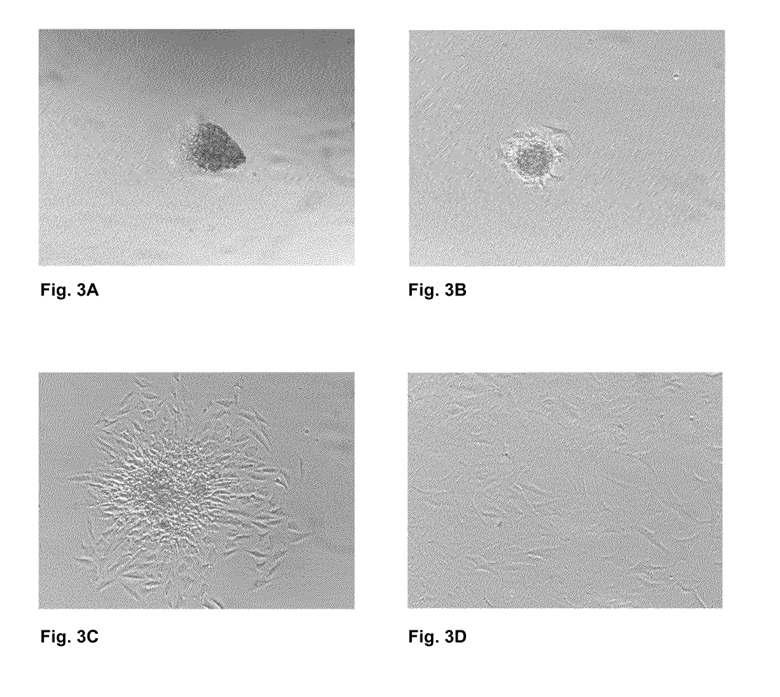

FIG. 3A shows a dermal papilla isolated by the enzymatic method described and placed in supplemented DMEM. FIG. 3B shows DP cells growing out of the attached dermal papilla 2 days after isolation and maintained in the supplemented DMEM. FIG. 3C shows DP cells after 7 days of culture in the same medium. FIG. 3D shows DP cells growing close to the complete confluence after 10 to 14 days of culture. All images were taken with a phase contrast microscope at 100X magnification.

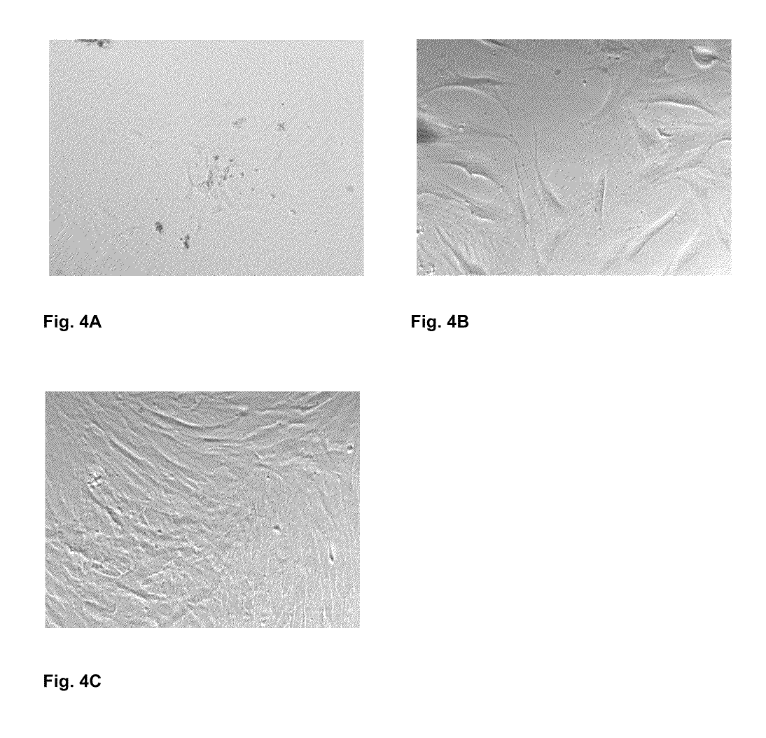

FIG. 4A shows DS cells 3 days after isolation by the enzymatic method described and placed in supplemented DMEM. FIG. 4B shows DS cells after 10 days of culture in the same medium. FIG. 4C shows DS cells growing to complete confluence after 20 days of culture. All images were taken with a phase contrast microscope at 100X magnification.

### Example 7: DP Sphere Formation

Cultured DP cells from confluent plates (see Example 6) were harvested and suspended in supplemented DMEM at a concentration of ˜10⁵ cells/ml. 100-μL aliquots of cell suspension were plated into each round-bottom well of a 96-well Low-Cell Binding Plate (Nunc). The DP spheres formed in one day and were maintained in the wells until they were used for implantation. DP spheres were collected and pulled in a concentration of 20 spheres in 100 μl DMEM supplemented with 10% FBS before being mixed with other cells.

### Example 8: Preparation the Mixture of Cells and Growth Promoting Cocktail in Hyaluronic Acid Gel

A cell mixture, containing 100 DP spheres and 10⁵ of each of DS, ORS, and EM cells were washed with PBS. The cell pellet was suspended in 250 μl PBS containing the following growth agents: IGF-1, FGF-2, FGF-10, PDGF-AA, Wnt-3a, Noggin, BMP-6, hypoxanthine, SHH, and Ephrin A3. 250 μl of hyaluronic acid gel (Q-Med AB) was added and mixed to the cells mixture. The mixture was mixed gently with a P1000 micropipette and drawn into a 1 cc syringe, while avoiding air bubbles by slowly and carefully collecting the liquid.

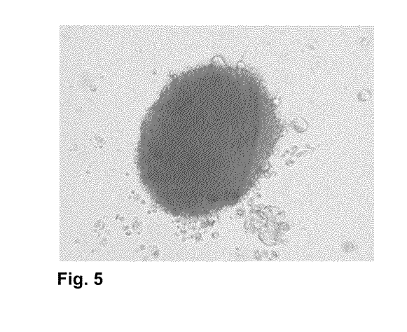

FIG. 5 shows a DP sphere formed from 10⁴ DP cells in a round-bottom well of a 96-well Low-Cell Binding Plate (Nunc) one day after seeding.

### Example 9: Cell Implantation to Human Scalp

The balding left and right temporal areas on the scalp of the volunteer (see Example 1) were indicated as the injection site, in which the number of existing hair follicles were counted by a trichometry device (TrichoScan or Folliscope). In the right temporal area, the mixture of cells and growth agents and hyaluronic acid gel (see Example 8) was used for a multiple injections under the skin. In the left temporal area, the mixture of cells and growth factors (i.e., without hyaluronic acid) was used for multiple injections under the skin (i.e between the epidermis and dermis). In both cases, 10 μl of mixture was delivered per injection.

The volunteer's temporal scalp was scanned with trichometry devices every two or three weeks for 3 months. At each visit, the injection areas were investigated for the total number of hairs, hair density per cm², as well as any pathologic symptoms.

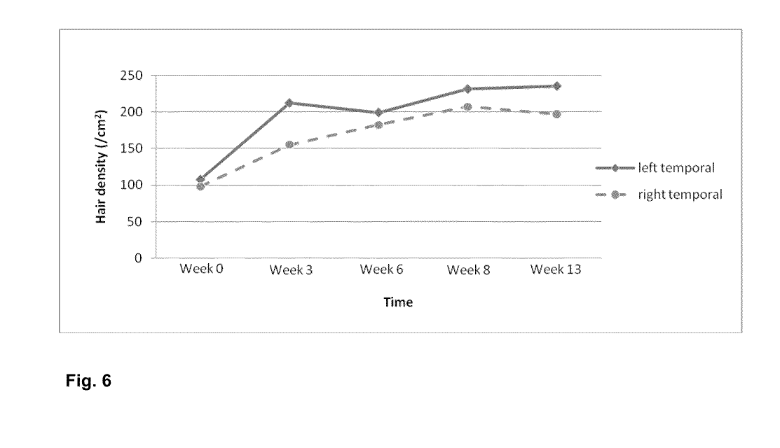

FIG. 6 shows a comparison of hair density per cm² in left temporal (red line), in which cells and growth factors were injected, with the hair density in right temporal (green line), in which cells and growth factors were mixed in hyaluronic acid gel before being injected. Both areas were scanned by using a Folliscope system before injections and on weeks 3, 6, and 8 post-injection.

The hair density in the left temporal area (receiving cells and growth factors only) showed a quick increase (96% compared to day 0) at week 3 post-injection; 3-6 weeks reaching a plateau; 6-8 weeks further increasing to reach 114% at week 8 post-injection (FIG. 6, solid line). The hair density in the right temporal area (receiving the mixture of cells and growth factors and hyaluronic acid gel) gradually increased by 58% at week 3, 86% at week 6, and 111% at week 8 post-injection (FIG. 6, dotted line).

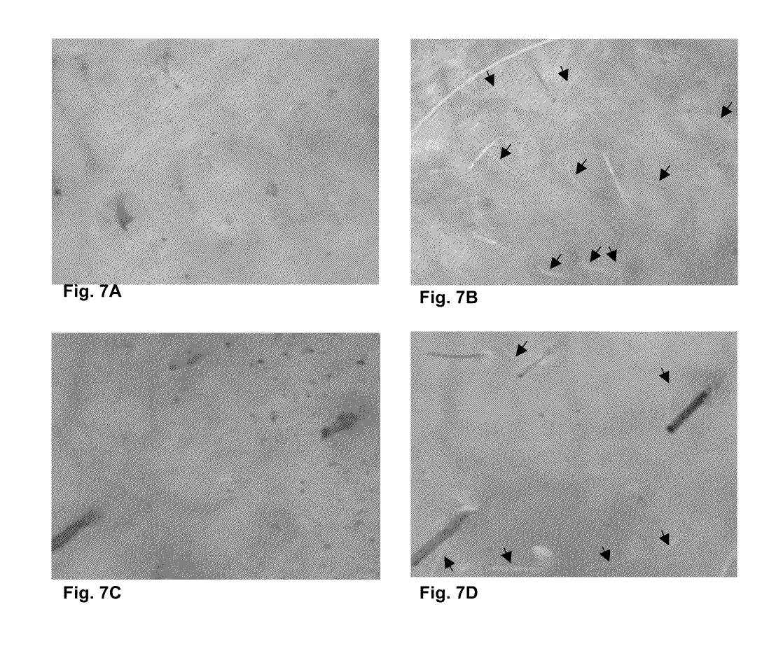

FIG. 7 depicts hair growth in the left temporal area (A and B), in which cells and growth factors were injected (without hyaluronic acid gel) compared to that of right temporal (C and D) areas, in which cells and growth factors were mixed in hyaluronic acid gel before being injected. The pictures were taken before the injections at week 0 (FIGS. 7A and 7C) and after 3 months (FIGS. 7B and 7D). In FIG. 7D, the hairs are stained for better visualization. The new hairs are indicated by arrows.

These results compare favourably to results reported in the prior art. For example, in United States Patent Application Publication 2007/0128172, when a mixture of 1.67×10⁵ epidermal cells, 2x10⁵ dermal papilla cells, and 1×10⁵ dermal sheath cells were transplanted to the forehead of a human subject, growth of only 3 hairs was observed after two weeks.

### REFERENCES

Botchkarev, V. A., and J. Kishimoto. 2003. Molecular control of epithelial-mesenchymal interactions during hair follicle cycling. The Journal of Investigative Dermatology. Symposium Proceedings/the Society for Investigative Dermatology, Inc.[and] European Society for Dermatological Research 8, (1) (Jun): 46-55.

Li, Y., C. M. Lin, X. N. Cai, and G. Q. Li. 2005. Reconstruction of human hair dermal papilla with microencapsulation in vitro. Journal of Dermatological Science 38, (2) (May): 107-9.

Oliver, R. F., and C. A. Jahoda. 1988. Dermal-epidermal interactions. Clinics in Dermatology 6, (4) (Oct-Dec): 74-82.

Oliver, R. F. 1991. Dermal-epidermal interactions and hair growth. The Journal of Investigative Dermatology 96, (5) (May): 76S.

Osada, A., T. Iwabuchi, J. Kishimoto, T. S. Hamazaki, and H. Okochi. 2007. Long-term culture of mouse vibrissal dermal papilla cells and de novo hair follicle induction. Tissue Engineering 13, (5) (May): 975-82.

Philpott, M., M. R. Green, and T. Kealey. 1989. Studies on the biochemistry and morphology of freshly isolated and maintained rat hair follicles. Journal of Cell Science 93 (Pt 3), (Pt 3) (Jul): 409-18.

Wu, J. J., T. Y. Zhu, Y. G. Lu, R. Q. Liu, Y. Mai, B. Cheng, Z. F. Lu, B. Y. Zhong, and S. Q. Tang. 2006. Hair follicle reformation induced by dermal papilla cells from human scalp skin. Archives of Dermatological Research 298, (4) (Sep): 183-90.

Yoo, B. Y., Y. H. Shin, H. H. Yoon, Y. J. Kim, K. Y. Song, S. J. Hwang, and J. K. Park. 2007. Improved isolation of outer root sheath cells from human hair follicles and their proliferation behavior under serum-free condition. Biotechnology and Bioprocess Engineering 12: 54-9.

We claim:

1. A method of generating a hair follicle in a scalp of a mammal, comprising administering a composition comprising epidermal matrix cells, dermal papilla cells, dermal sheath cells and outer root sheath cells to the scalp.

2. The method of claim 1, wherein the ratio of dermal papilla cells to dermal sheath cells is 15:1 to 1:1, the ratio of outer root sheath cells to epidermal matrix cells is 15:1 to 1:1, and the ratio of dermal papilla cells plus dermal sheath cells to outer root sheath cells plus epidermal matrix cells is 10:1 to 0.5:1.

3. The method of claim 1, wherein the composition comprises:
1-3×10⁴ dermal papilla cells,
1-3×10⁴ outer root sheath cells,
1-3×10³ dermal sheath cells, and
1-3×10³ epidermal matrix cells.

4. The method of claim 1, wherein the cells are isolated from a native hair follicle.

5. The method of claim 1, wherein each type of cell of the composition is expanded separately in vitro prior to administration.

6. The method of claim 5, wherein each type of cell is expanded in media specific to the cell.

7. The method of claim 5, wherein the epidermal matrix cells are expanded in medium comprising antibiotic, antimycotic, human recombinant bFGF, human insulin, hydrocortisone and bovine pituitary extract.

8. The method of claim 5, wherein the outer root sheath cells are expanded in medium comprising antibiotic, antimycotic, human recombinant EGF, human insulin, hydrocortisone, epinephrine, human transferrin and bovine pituitary extract.

9. The method of claim 5, wherein the dermal papilla cells are expanded in medium comprising FBS, L-glutamine, antibiotic, antimycotic, human recombinant bFGF, human recombinant Wnt-3 and human recombinant BMP6.

10. The method of claim 5, wherein the dermal sheath cells are expanded in medium comprising FBS, L-glutamine, antibiotic, antimycotic, human recombinant bFGF and human recombinant PDGF-AA.

11. The method of claim 1, wherein the dermal papilla cells are aggregated into at least one sphere, the at least one sphere optionally formed by microencapsulation or centrifugation.

12. The method of claim 11, wherein there are 100 to 100,000 dermal papilla cells in the at least one sphere.

13. The method of claim 11, wherein the at least one sphere further comprises dermal sheath, epidermal matrix and/or outer root sheath cells.

14. The method of claim 1, wherein the composition is administered to a native hair follicle in the scalp, such that the cells contact the native hair follicle.

15. The method of claim 1, wherein the composition is administered to an incision in the scalp.

16. The method of claim 1, wherein the composition is administered to the scalp between the dermis and the epidermis.

17. The method of claim 1, wherein the composition further comprises at least one hair growth agent, the at least one hair growth agent selected from the group consisting of: IGF-1, FGF-2, FGF-10, PDGF-AA, Wnt-3a, noggin, ephrin-A3, SHH, BMP-6 and hypoxanthine.

18. The method of claim 1, wherein the composition is administered by injection into the scalp.

19. The method of claim 18, wherein each injection contains 1 microliter to 100 microliters of the composition.

20. The method of claim 1, wherein the composition is incorporated in a carrier prior to administration.

21. A composition comprising epidermal matrix cells, dermal papilla cells, dermal sheath cells and outer root sheath cells.

22. The composition of claim 21, wherein the ratio of dermal papilla cells to dermal sheath cells is 15:1 to 1:1, the ratio of outer root sheath cells to epidermal matrix cells is 15:1 to 1:1, and the ratio of dermal papilla cells plus dermal sheath cells to outer root sheath cells plus epidermal matrix cells is 10:1 to 0.5:1.

23. The composition of claim 21, wherein the composition comprises
1-3×10⁴ dermal papilla cells,
1-3×10⁴ outer root sheath cells,
1-3×10³ dermal sheath cells, and
1-3×10³ epidermal matrix cells.

24. The composition of claim 21, wherein the composition further comprises at least one hair growth agent, the hair growth agent optionally selected from the group consisting: IGF-1, FGF-2, FGF-10, PDGF-AA, Wnt-3a, noggin, ephrin-A3, SHH, BMP-6 and hypoxanthine.

25. The composition of claim 21, wherein the composition further comprises IGF-1, FGF-2, FGF-10, PDGF-AA, Wnt-3a, noggin, ephrin-A3, SHH, BMP-6 and hypoxanthine.

26. The composition of claim 21, wherein the composition comprises a carrier, optionally a biomatrix.
